(19) United States
(12) Patent Application Publication
(10) Pub. No.: US 2005/0171126 A1
(43) Pub. Date: Aug. 4, 2005

(54) Process for the production of purine nucleoside compounds

(75) Inventors: Takayoshi Torii, Kawasaki-shi (JP); Tomoyuki Onishi, Kawasaki-shi (JP); Kunisuke Izawa, Kawasaki-shi (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: Ajinomoto Co., Inc., Tokyo (JP)

(21) Appl. No.: 11/016,741

(22) Filed: Dec. 21, 2004

(30) Foreign Application Priority Data
Dec. 26, 2003 (JP) 2003-434009

(51) Int. Cl. A61K 31/52; C07D 473/10

(52) U.S. Cl. 514/263.23; 544/277

(57) ABSTRACT

2′,3′-didehydro-2′,3′-dideoxypurine nucleoside compounds and 2′,3′-dideoxypurine nucleoside compounds may be produced efficiently by treating a 3′-deoxy-3′-bromopurine nucleoside compound with a perfluoroalkanesulfonyl fluoride in the presence of a base to give a 2′,3′-didehydro-2′,3′-dideoxypurine nucleoside compound, which may be converted to a 2′,3′-dideoxypurine nucleoside compound, by catalytic hydrogenation.

### CROSS REFERENCES TO RELATED APPLICATIONS

This claims priority to Japanese Patent Application No. 434009/2003, filed on Dec. 26, 2003, which is incorporated herein by reference in its entirety.

### BACKGROUND OF THE INVENTION

1. Field of the Invention

The present invention relates to processes for the production of 2′,3′-didehydro-2′,3′-dideoxypurine nucleoside compounds and 2′,3′-dideoxoypurine nucleoside compounds. The present invention further relates to intermediate compounds which are useful for the production of such compounds.

2. Discussion of the Background

2′,3′-Didehydro-2′,3′-dideoxypurine nucleoside compounds (hereinafter, sometimes referred to as a D4 compound) represented by cyclo D4G (see, WO 02/062,123) and 2′,3′-dideoxypurine nucleoside compounds (hereinafter, sometimes referred to as a DD compound) represented by DDI are useful as pharmaceuticals, e.g., such as anti-HIV drugs.

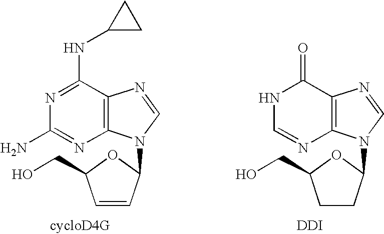

A process for the production of a D4 compound in which an acetoxybromo derivative of a purine nucleoside is reduced with zinc is reported in Tetrahedron Letters, (England), vol. 25, pp. 367-370 (1984). However, in this method, it is necessary to add zinc metal in more than a stoichiometric amount, and it is difficult to remove zinc from the reaction solution after completion of the reaction. In addition, a large amount of waste zinc is produced, this method is not preferred in view of environment concerns.

Further, a method in which a 2′,3′-dithiocarbonyl derivative of a purine nucleoside is subjected to a radical reduction is reported in Journal of Organic Chemistry, (U.S.A.), vol. 54, pp. 2217-2225 (1989). However, since a radical reduction is carried out in this reaction, it is necessary that protection and deprotection of a hydroxyl group are carried and there is the problem that the yield of the desired product is significantly lowered.

Thus, there remains a need for improved methods for the production of 2′,3′-didehydro-2′,3′-dideoxypurine nucleoside compounds and 2′,3′-dideoxoypurine nucleoside compounds. There also remains a need for intermediate compounds which are useful in such methods.

### SUMMARY OF THE INVENTION

Accordingly, it is one object of the present invention to provide novel processes by which a 2′,3′-didehydro-2′,3′-dideoxypurine nucleoside compound and a 2′,3′-dideoxypurine nucleoside compound are efficiently produced.

It is another object of the present invention to provide novel intermediates which are useful for producing such compounds in such processes.

These and other objects, which will become apparent during the following detailed description, have been achieved by the inventors' discovery that that, when a 3′-deoxy-3′-halopurine nucleoside compound is treated with a perfluoroalkanesulfonyl fluoride in the presence of a base, the corresponding 2′,3′-didehydro-2′,3′-dideoxypurine nucleoside may be obtained in a single step, in a high yield. The resulting 2′,3′-didehydro-2′,3′-dideoxypurine nucleoside compound may then be easily converted into the corresponding 2′,3′-dideoxypurine nucleoside when the double bond is subjected to catalytic hydrogenation.

Thus, the present invention provides:

(1) A method for the production of a 2′,3′-didehydro-2′,3′-dideoxypurine nucleoside compound represented by formula (2): 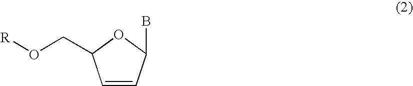
wherein:
R is a protective group for a hydroxyl group; and
B is a purine base,
   wherein said method comprises:
(a) treating a 3′-deoxy-3′-halopurine nucleoside compound represented by formula (1): 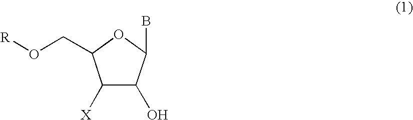
   wherein:
X is chlorine atom, bromine atom, or iodine atom; and
R and B are defined above,
with a perfluoroalkanesulfonyl fluoride in the presence of a base, to obtain said 2′,3′-didehydro-2′,3′-dideoxypurine nucleoside compound of formula (2).

(2) A method for the production of a 2′,3′-dideoxypurine nucleoside compound represented by formula (3): 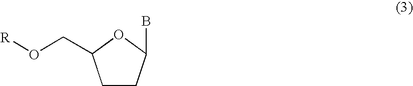
wherein:
R is a protective group for a hydroxyl group; and
B is a purine base,
   wherein said method comprises:
(a) treating a 3′-deoxy-3′-halopurine nucleoside compound represented by formula (1): 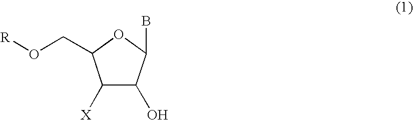
   wherein:
X is chlorine atom, bromine atom or iodine atom; and
R and B are defined above,
   with a perfluoroalkanesulfonyl fluoride in the presence of a base, to obtain a 2′,3′-didehydro-2′,3′-dideoxypurine nucleoside compound represented by formula (2): 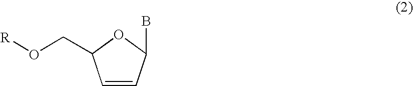
   wherein:
R and B are defined above; and
(b) subjecting said 2′,3′-didehydro-2′,3′-dideoxypurine nucleoside compound of formula (2) to catalytic hydrogenation.

(3) The method according to (1) or (2), wherein the base is a tertiary amine.

(4) A method for the production of a 2′,3′-didehydro-2′,3′-dideoxypurine nucleoside compound represented by formula (2′): 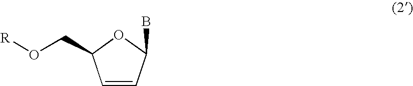
wherein:
R is a protective group for a hydroxyl group; and
B is a purine base,
   wherein said method comprises:
(a) treating a 3′-deoxy-3′β-halopurine nucleoside compound represented by formula (1′): 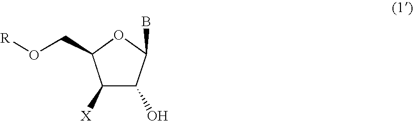
   wherein:
X is chlorine atom, bromine atom, or iodine atom; and
R and B are defined above, treated with a perfluoroalkanesulfonyl fluoride in the presence of a base, to obtain said 2′,3′-didehydro-2′,3′-dideoxypurine nucleoside compound of formula (2′).

(5) A method for the production of a 2′,3′-dideoxypurine nucleoside compound represented by formula (3′): 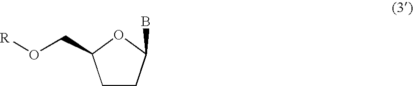
wherein: wherein:
R is a protective group for a hydroxyl group; and
B is a purine base,
   wherein said method comprises:
(a) treating a 3′-deoxy-3′β-halopurine nucleoside compound represented by formula (1′): 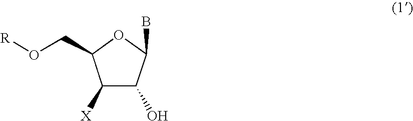
   wherein:
X is chlorine atom, bromine atom, or iodine atom; and
   R and B are defined above, treated with a perfluoroalkanesulfonyl fluoride in the presence of a base, to obtain said 2′,3′-didehydro-2′,3′-dideoxypurine nucleoside compound of formula (2′): 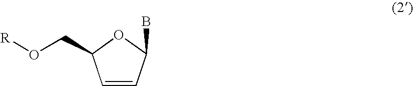
   wherein:
R and B are defined above; and
(b) subjecting said 2′,3′-didehydro-2′,3′-dideoxypurine nucleoside compound of formula (2′) to catalytic hydrogenation.

(6) The method according to (4) or (5), wherein the base is a tertiary amine.

(7) N²,5′-O-Diacetyl-3′-deoxy-3′β-bromoguanosine represented by formula (1a): 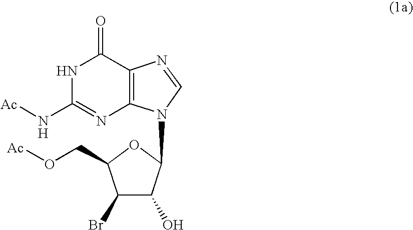
wherein Ac is acetyl group.

(8) N²,5′-O-Diacetyl-2′,3′-didehydro-2′,3′-dideoxy-guanosine represented by formula (2a): 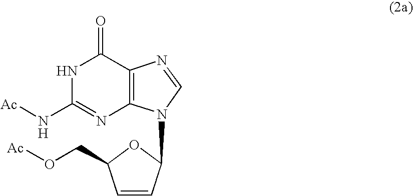
wherein Ac is acetyl group.

(9) N²,5′-O-Diacetyl-3′-deoxy-3′β-bromo-2-amino-6-chloropurine riboside represented by formula (1b): 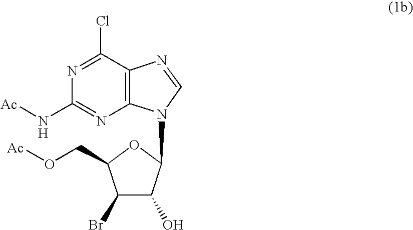
wherein Ac is acetyl group.

(10) N²,5′-O-Diacetyl-2′,3′-didehydro-2′,3′-dideoxy-2-amino-6-chloropurine riboside represented by formula (2b): 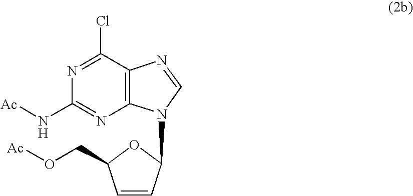
wherein Ac is acetyl group.

(11) A method for producing a 2′,3′-didehydro-2′,3′-dideoxypurine nucleoside compound represented by formula (6): 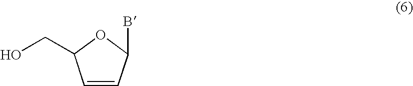
wherein:
   B′ is a purine base,
wherein said method comprises:
(a) treating a 3′-deoxy-3′-halopurine nucleoside compound represented by formula (1): 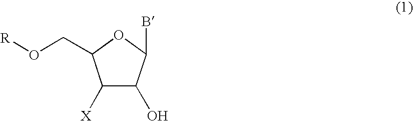
wherein:
   X is chlorine atom, bromine atom, or iodine atom;
   R is a protecting group; and
   B′ is defined above,
   with at least one perfluoroalkanesulfonyl fluoride in the presence of at least one base, to obtain a 2′,3′-didehydro-2′,3′-dideoxypurine nucleoside compound represented by formula (2): 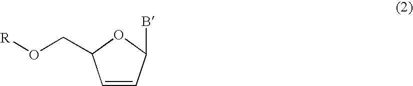
wherein
   R and B′ are defined above; and
(b) subjecting said 2′,3′-didehydro-2′,3′-dideoxypurine nucleoside compound of formula (2) to de-protection of R, and if necessary to at least one of protection, de-protection, and modification of a group at 2-position and/or 6-position of the purine base.

(12) A method for producing a 2′,3′-didehydro-2′,3′-dideoxypurine nucleoside compound represented by formula (6′): 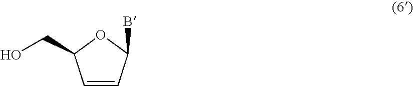
wherein:
   B′ is a purine base,
wherein said method comprises:
(a) treating a 3′-deoxy-3′-halopurine nucleoside compound represented by formula (1′): 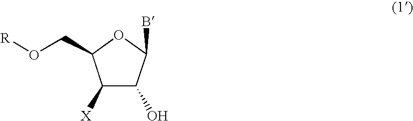
wherein
   X is chlorine atom, bromine atom, or iodine atom;
   R is a protecting group; and
   B′ is defined above,
   with at least one perfluoroalkanesulfonyl fluoride in the presence of at least one base, to obtain a 2′,3′-didehydro-2′,3′-dideoxypurine nucleoside compound represented by formula (2′) 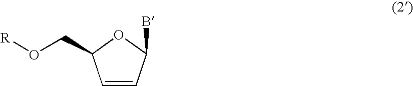
wherein:
   R and B′ are defined above; and
(b) subjecting said 2′,3′-didehydro-2′,3′-dideoxypurine nucleoside compound of formula (2′) to de-protection of R, and if necessary to at least one of protection, de-protection, and modification of a group at 2-position and/or 6-position of the purine base.

(13) A method for producing a 2′,3′-dideoxypurine nucleoside compound represented by formula (7): 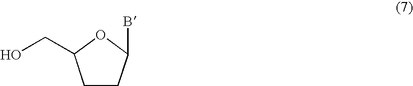
wherein:
   B′ is a purine base,
wherein said method comprises:
(a) treating a 3′-deoxy-3′-halopurine nucleoside compound represented by formula (1): 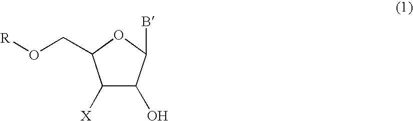
wherein:
   X is chlorine atom, bromine atom, or iodine atom;
   R is a protecting group; and
   B′ is defined above,
   with at least one perfluoroalkanesulfonyl fluoride in the presence of at least one base, to obtain a 2′,3′-didehydro-2′,3′-dideoxypurine nucleoside compound represented by formula (2): 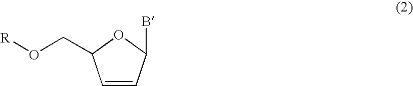
wherein
   R and B′ are defined above;
(b) subjecting said 2′,3′-didehydro-2′,3′-dideoxypurine nucleoside compound of formula (2) to catalytic hydrogenation, to obtain a 2′,3′-dideoxypurine nucleoside compound of formula (3): 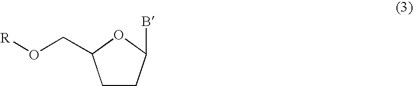
wherein:
   R and B′ are defined above; and
(c) subjecting said 2′,3′-dideoxypurine nucleoside compound of formula (3) to de-protection of R, and if necessary to at least one of protection, de-protection, and modification of a group at 2-position and/or 6-position of the purine base.

(14) A method for producing a 2′,3′-dideoxypurine nucleoside compound represented by formula (7′) 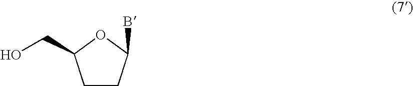
wherein:
   B′ is a purine base,
wherein said method comprises:
(a) treating a 3′-deoxy-3′-halopurine nucleoside compound represented by formula (1′): 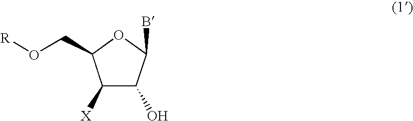
wherein:
   X is chlorine atom, bromine atom, or iodine atom;
   R is a protecting group; and
   B′ is defined above,
   with at least one perfluoroalkanesulfonyl fluoride in the presence of at least one base, to obtain a 2′,3′-didehydro-2′,3′-dideoxypurine nucleoside compound represented by formula (2′): 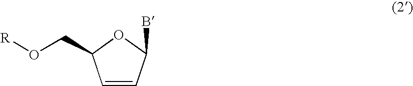
wherein:
   R and B′ are defined above;
(b) subjecting said 2′,3′-didehydro-2′,3′-dideoxypurine nucleoside compound of formula (2′) to catalytic hydrogenation, to obtain a 2′,3′-dideoxypurine nucleoside compound of formula (3′): 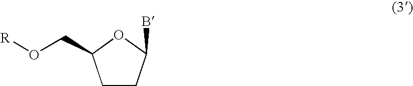
wherein:
   R and B′ are defined above; and
(c) subjecting said 2′,3′-dideoxypurine nucleoside compound of formula (3′) to de-protection of R, and if necessary to at least one of protection, de-protection, and modification of a group at 2-position and/or 6-position of the purine base.

In accordance with the present invention, it is possible to efficiently produce 2′,3′-didehydro-2′,3′-dideoxypurine nucleoside compounds and 2′,3′-dideoxypurine nucleoside compounds which are useful as pharmaceuticals or intermediates therefor.

### DETAILED DESCRIPTION OF THE PREFERRED EMBODIMENTS

In the formulae of the present invention, X is chlorine atom, bromine atom, or iodine atom; R is a protective group for a hydroxyl group; and B is a purine base.

There is no particular limitation for the protective group for a hydroxyl group and its examples include an acyl group, an alkyl group, an aralkyl group, and a silyl group. Examples of the acyl group are acyl groups having 1 to 7 carbon(s) such as a formyl group, an acetyl group, a pivaloyl group, and a benzoyl group. Examples of the alkyl group are alkyl groups having 1 to 7 carbons such as a methyl group, an ethyl group, a n-propyl group, an isopropyl group, a n-butyl group, and a tert-butyl group. Examples of the aralkyl group are aralkyl groups having 7 to 22 carbons such as a benzyl group, a trityl group, a 4-monomethoxytrityl group, and a 4,4′-dimethoxytrityl group. Examples of the silyl group are tri-substituted silyl groups such as a trimethylsilyl group, a triethylsilyl group, and a tert-butyldimethylsilyl group. Besides those, it is possible to use a protective group for hydroxyl groups such as a methoxymethyl group, a methylthiomethyl group, a benzyloxymethyl group, a methoxyethoxymethyl group, a tetrahydropyranyl group, a methoxycarbonyl group, a 9-fluorenylmethoxycarbonyl group, a 2,2,2-trichloroethoxycarbonyl group, a benzyloxycarbonyl group, and a tert-butoxycarbonyl group. Preferred protective groups for a hydroxyl group are an acetyl group, a benzoyl group, a benzyl group, and a trityl group, and an acetyl group is particularly preferred.

An example of the purine base is a purine base represented by the following formula (4): 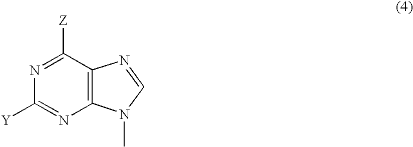
wherein Y and Z may be same or different and each is an optionally substituted amino group, an optionally substituted hydroxyl group, a halogen atom, or a hydrogen atom.

Examples of the optionally substituted amino group include both mono- and di-substituted amino groups, as well as an unsubstituted amino group. Examples of the substituents when the amino group is substituted include one or two of the following substituents: an alkyl group, an aralkyl group, an acyl group, and a carbamoyl group. Examples of the alkyl group include alkyl groups having 1 to 10 carbon(s) such as a methyl group, an ethyl group, a n-propyl group, an isopropyl group, a n-butyl group, and a tert-butyl group. Examples of the aralkyl group include aralkyl groups having 7 to 22 carbons such as a benzyl group, a trityl group, a 4-monomethoxytrityl group, and a 4,4′-dimethoxytrityl group. Examples of the acyl group include acyl groups having 1 to 10 carbon(s) such as a formyl group, an acetyl group, a pivaloyl group, and a benzoyl group. Examples of the carbamoyl group include carbamoyl groups such as a benzyloxycarbonyl group, a tert-butoxycarbonyl group, and a methoxycarbonyl group.

Examples of the substituent when the hydroxyl group is substituted include the same ones already mentioned as the protective groups for a hydroxyl group.

Examples of the halogen atom are chlorine atom, fluorine atom, bromine atom, and iodine atom.

Specific examples of the purine base include purine, adenine, guanine, N-acetylguanine, hypoxanthine, xanthine, 6-fluoropurine, 6-chloropurine, 6-methylaminopurine, 6-dimethylaminopurine, 6-trifluoromethylaminopurine, 6-cyclopropylaminopurine, 6-benzoylaminopurine, 6-acetylaminopurine, 6-methoxypurine, 6-acetoxypurine, 6-benzoyloxypurine, 6-methylpurine, 6-ethylpurine, 6-trifluoromethylpurine, 6-phenylpurine, 6-mercaptopurine, 6-methylmercaptopurine, 6-aminopurine-1-oxide, 6-hydroxypurine-1-oxide, 2,6-diaminopurine, 2-amino-6-chloropurine, 2-acetylamino-6-chloropurine, 2-aminopurine, 2-amino-6-mercaptopurine, 2-amino-6-methylmercaptopurine, 2-amino-6-hydroxyaminopurine, 2-amino-6-methoxypurine, 2-amino-6-benzoyloxypurine, 2-amino-6-acetoxypurine, 2-amino-6-methylpurine, 2-amino-6-cyclopropylaminopurine, and 2-amino-6-phenylpurine.

Preferred purine bases include adenine, guanine, N-acetylguanine, hypoxanthine, 2-amino-6-chloropurine, 2-acetylamino-6-chloropurine, and 2-amino-6-cyclopropylaminopurine. Particularly preferred ones include adenine, N-acetylguanine, hypoxanthine, and 2-acetylamino-6-chloropurine.

The method for the production of a 2′,3′-didehydro-2′,3′-dideoxypurine nucleoside compound represented by formula (2) according to the present invention is characterized in that a 3′-deoxy-3′-bromopurine nucleoside compound represented by the formula (1) is treated with a perfluoroalkanesulfonyl fluoride in the presence of a base.

Examples of the base include alkaline metal hydroxides such as sodium hydroxide, potassium hydroxide, and lithium hydroxide; alkaline earth metal hydroxides such as calcium hydroxide; alkaline metal carbonates such as sodium carbonate and potassium carbonate; alkaline earth metal carbonates such as calcium carbonate; alkaline metal hydrogen carbonates such as sodium hydrogen carbonate; and tertiary amines such as trimethylamine, triethylamine, triethylenediamine, N,N-dimethylcyclohexylamine, tetramethylenediamine, N,N,N′,N′-tetramethyl-1,3-butanediamine, N-methylmorpholine, N,N-diethyl-2-methylpiperazine, and DBU (1,8-diazabicyclo[5,4,0]undec-7-ene). Tertiary amines are used particularly advantageously.

With regard to the perfluoroalkanesulfonyl fluoride, a perfluoroalkanesulfonyl fluoride represented by the following formula (5) may be exemplified.
P—SO₂—F (5)
wherein P is a perfluoroalkyl group.

With regard to the perfluoroalkyl group, a perfluoroalkyl group having 1 to 10 carbon(s) may be preferably exemplified. Specific examples of the preferred perfluoroalkanesulfonyl fluoride include perfluorobutanesulfonyl fluoride, perfluorohexanesulfonyl fluoride, and perfluorooctanesulfonyl fluoride. Perfluorobutanesulfonyl fluoride is particularly preferred.

Although there is no particular limitation for the amount of the base used, it is preferably used in an amount from 1 to 10 or, more preferably from 2 to 5, in terms of a molar ratio to 1 mole of the 3′-deoxy-3′-halopurine nucleoside compound.

Although there is no particular limitation for the amount of the perfluoroalkanesulfonyl fluoride used, it is preferably used in an amout from 1 to 10 or, more preferably from 1 to 5, in terms of a molar ratio to 1 mol of the 3′-deoxy-3′-halopurine nucleoside compound.

Although there is no particular limitation for the ratio of the base to the perfluoroalkanesulfonyl fluoride, it is preferred that, in terms of a molar ratio, the base is used in an amount from 0.5 to 10 or, more preferably from 1 to 5, relative to 1 mol of the perfluoroalkanesulfonyl fluoride.

With regard to a solvent used for the reaction, an aprotic organic solvent may be used and, for example, tetrahydrofuran, dioxane, ethyl acetate, dichloromethane, chloroform, toluene, hexane, acetonitrile, N,N-dimethylformamide, N,N-dimethylacetamide, or a mixed solvent of any of them may be used.

Although there is no particular limitation for the concentration of the 3′-deoxy-3′-halopurine nucleoside compound in the reaction solvent at the start of the reaction, it is preferably from 0.05 to 1.0 mol/L or, more preferably, from 0.1 to 0.5 mol/L.

Although there is no particular limitation for the reaction temperature, it may be carried out usually within a range of 20 to 120° C. and, preferably, 50 to 80° C. The reaction time is not particularly limited as well, and it may be usually carried out for 0.1 to 10 hour(s) and, preferably, 1 to 5 hour(s).

After completion of the reaction, the desired substance may be isolated and purified by any appropriate use of operations which have been known by persons skilled in the art such as extraction, crystallization, and chromatography.

When the resulting 2′,3′-didehydro-2′,3′-dideoxypurine nucleoside compound is subjected to a catalytic hydrogenation, it is converted into a 2′,3′-dideoxypurine nucleoside compound.

The catalytic hydrogenation may be carried out in such a manner that 0.1 to 10 atmosphere(s) (10 to 1,000 kilopascal(s)) of hydrogen gas is made to react for 0.1 to 24 hour(s) at a reaction temperature of 0 to 80° C., in a solvent such as methanol, acetonitrile, water, and N,N-dimethylformamide, and in the presence of a catalyst such as palladium carbon, rhodium carbon, and ruthenium carbon. The catalyst may be removed from the resulting reaction solution by filtration, and the desired substance may be obtained by any method which has been known by persons skilled in the art such as evaporation of the solvent, crystallization, and chromatography.

The compound of formula (1) which is a starting substance may be easily prepared according to the method mentioned in Nucleosides & Nucleotides, vol. 15, pp. 31 to 45 (1996).

As for the 3′-deoxy-3′-halopurine nucleoside compound represented by formula (1) in the present invention, a 3′-deoxy-3′β-halopurine nucleoside compound represented by the following formula (1′) may be advantageously used. 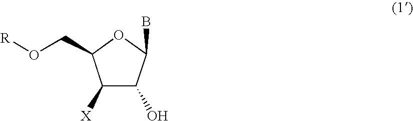
wherein X is chlorine atom, bromine atom, or iodine atom; R is a protective group for a hydroxyl group; and B is a purine base.

The symbols X, R and B in the formula are the same as those mentioned already.

As mentioned already, the 3′-deoxy-3′β-halopurine nucleoside compound represented by the formula (1′) may be converted into a 2′,3′-didehydro-2′,3′-dideoxypurine nucleoside compound represented by the following formula (2′) by treatment with a perfluoroalkanesulfonyl fluoride in the presence of a base according to the present invention. 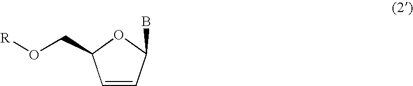
wherein R and B have the same meanings as defined already.

Further, the 2′,3′-didehydro-2′,3′-dideoxypurine nucleoside compound represented by formula (2′) may be converted into a 2′,3′-dideoxypurine nucleoside compound represented by formula (3′) by catalytic hydrogenation. 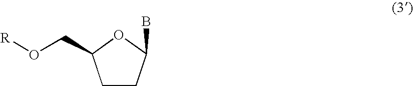
wherein R and B have the same meanings as defined already.

Within the compounds of the formula (1′), N²,5′-O-diacetyl-3′-deoxy-3′β-bromoguanosine represented by the following formula (1a) and N²,5′-O-diacetyl-3′-deoxy-3′β-bromo-2-amino-6-chloropurine riboside represented by the following formula (1b) are novel substances. 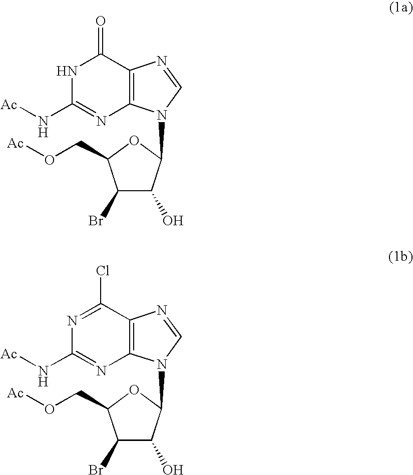
In each formula, Ac is acetyl group.

Further, within the compounds of the formula (2′), N²,5′-O-diacetyl-2′,3′-didehydroguanosine represented by the following formula (2a) and N²,5′-O-diacetyl-2′,3′-didehydro-2′,3′-dideoxy-2-amino-6-chloropurine riboside represented by the following formula (2b) are novel substances. 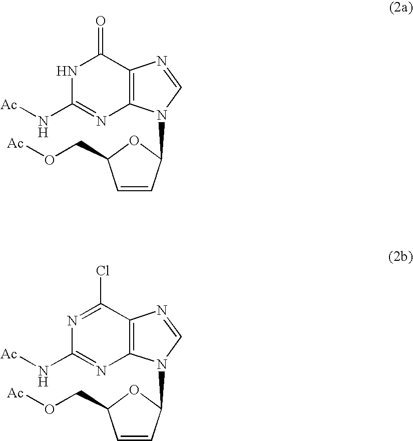
In each formula, Ac is acetyl group.

The 2′,3′-didehydro-2′,3′-dideoxypurine nucleoside compounds represented by formula (2) and formula (2′) may be converted into 2′,3′-didehydro-2′,3′-dideoxypurine nucleoside compounds represented by formula (6) and formula (6′) respectively by subjecting compound (2) or (2′) to de-protection of R, and if necessary to at least one of protection, de-protection, and modification of a group at the 2-position and/or the 6-position of the purine base.

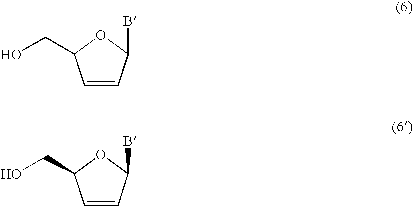

In formulae (6) and (6′), B′ is a purine base. The purine base is the same as described above.

Furthermore, the 2′,3′-dideoxypurine nucleoside compounds represented by formula (3) and formula (3′) may be converted into 2′,3′-dideoxypurine nucleoside compounds represented by formula (7) and formula (7′) respectively by subjecting compound (3) or (3′) to de-protection of R, and if necessary to at least one of protection, de-protection, and modification of a group at the 2-position and/or the 6-position of the purine base.

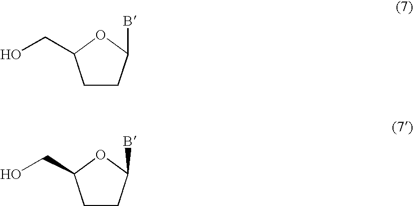

In formulae (7) and (7′), B′ is a purine base. The purine base is the same as described above.

The “protecting group” means a group which is intended to be removed after performing the desired conversion of another part of the compound. The de-protection of protecting group R for hydroxyl group may be performed using the methods described in, for example, Nucleoside & Nucleotides, vol. 7(2), pp. 143-153 (1998), Tetrahedron Letters, vol. 29(11), pp. 1239-1243 (1988), and the like.

The groups at the 2-position and the 6-position of the purine base correspond to Y and Z in formula (4) respectively. “If necessary” means the case that group at the 2-position and/or the 6-position of the purine base is converted to another group.

“Protection” of the 2-position and/or the 6-position of the purine base includes, for example, a step of introducing a protecting group exemplified above when a group at the 2-position and/or the 6-position of the purine base is an amino group and/or a hydroxyl group. The said “protection” and “de-protection” may be performed by well known conventional methods (for example, see, Protective Groups in Organic Synthesis, 3rd edn., Wiley Interscience Publication, John Wiley & Sons, Inc., 1999).

“Modification” means a conversion of group which is different from the above described “protection” and “de-protection.” For example, “Modification” means a step of conversion to an optional group such as a halogen atom or a hydrogen atom when the group at the 2-position and/or the 6-position of the purine base is an amino group and/or a hydroxyl group. “Modification” may be performed by well known conventional methods in the synthesis of nucleic acids (for example, see, Nucleic acid Chemistry Part II, Leroy B. Townsend, R. Stuart Tipson, A Wiley-Interscience Publication).

Other features of the invention will become apparent in the course of the following descriptions of exemplary embodiments which are given for illustration of the invention and are not intended to be limiting thereof.

### EXAMPLES

### Reference Example 1

### Production of N²,5′-O-diacetyl-3′-deoxy-3′β-bromoguanosine

To a suspension of N²,2′,5′-O-triacetyl-3′-deoxy-3′β-bromoguanosine (944 mg, 2.0 mmol) in ethanol (20 mL) were added hydroxylamine hydrochloride (278 mg, 4.0 mmol) and triethylamine (0.558 mL, 4.0 mmol) followed by stirring for 14 hours at room temperature. The solid was removed from the resulting suspension by filtration and dried in vacuo to give the desired substance (618 mg, yield: 72%) as a white solid.

¹H-NMR (DMSO-d6): δ 2.06 (s, 3H), δ 2.19 (s, 3H, δ 4.32-4.40 (m, 2H), δ 4.50-4.57 (m, 1H), δ 4.63 (d-d, 1H, J=5.2, 3.7 Hz), δ 4.83-4.88 (m, 1H), δ 5.75 (d, 1H, J=3.8 Hz), δ 6.51-6.55 (m, 1H), δ 8.18 (s, 1H), δ 11.77 (br, 1H), δ 12.10 (br, 1H).

ESIMS m/z: 430 (M+H)

### Reference Example 2

### Production of N²,5′-O-diacetyl-3′-deoxy-3′β-bromo-2-amino-6-chloropurine Riboside

To a suspension of N²,2′,5′-O-triacetyl-3′-deoxy-3′β-bromo-2-amino-6-chloropurine riboside (260 mg, 0.5 mmol) in ethanol (5 mL) were added hydroxylamine hydrochloride (70 mg, 1.0 mmol) and triethylamine (0.14 mL, 1.0 mmol) followed by stirring for 14 hours at room temperature, and the resulting solution was concentrated. The desired substance (110 mg, yield: 49%) as white solid was obtain by purification by means of chromatography (15 g of silica gel; eluted with hexane-ethyl acetate in 1:2) and drying in vacuo.

¹H-NMR (CDCl₃): δ 1.81 (s, 3H), δ 2.29 (s, 3H), δ 4.35-4.41 (m, 1H), δ 4.49 (d-d, 1H, J=12.4, 3.3 Hz), δ 4.53-4.58 (m, 1H), δ 4.72-4.78 (m, 1H), δ 4.91-4.96 (m, 1H), δ 5.87 (d, 1H, J=4.3 Hz), δ 6.26 (br, 1H), δ 8.22 (d, 1H, J=3.6 Hz), δ 8.35 (br, 1H).

ESIMS m/z: 448 (M+M).

### Example 1

### Production of 5′-O-acetyl-2′,3′-didehydro-2′,3′-dideoxyadenosine

To a solution of 5′-O-acetyl-3′-deoxy-3′β-bromo-adenosine (186 mg, 0.5 mmol) in acetonitrile (5 mL) were added triethylamine (0.278 mL, 2.0 mmol) and perfluoro-1-butanesulfonyl fluoride (0.360 mL, 2.0 mmol) followed by stirring for 2 hours with heating at 70° C. The resulting solution was cooled down to room temperature and concentrated in vacuo. As a result of purification by chromatography (20 g of silica gel; eluted with dichloromethane-methanol in 32:1), the desired substance (135 mg, yield: 98%) was obtained as a white solid.

¹H-NMR (DMSO-d6): δ 1.99 (s, 3H), δ 4.14-4.21 (m, 2H), δ 5.07-5.12 (m, 1H), δ 6.23-6.28 (m, 1H), δ 6.47-6.52 (m, 1H), δ 6.93-6.97 (m, 1H), δ 7.29 (br, 2H), δ 8.08 (s, 1H), δ 8.17 (s, 1H).

ESIMS m/z: 276 (M+H).

### Example 2

### Production of N²,5′-O-diacetyl-2′,3′-didehydro-2′,3′-dideoxyguanosine

To a solution of N²,5′-O-diacetyl-3′-deoxy-3′β-bromo-guanosine (215 mg, 0.5 mmol) in acetonitrile (5 mL) were added triethylamine (0.278 mL, 2.0 mmol) and perfluoro-1-butane-sulfonyl fluoride (0.360 mL, 2.0 mmol) followed by heating at 70° C. for 2 hours with stirring. The resulting solution was cooled down to room temperature and concentrated in vacuo. As a result of purification by chromatography (20 g of silica gel; eluted with dichloromethane-methanol in 32:1), the desired substance (104 mg, yield: 62%) was obtained as a white solid.

¹H-NMR (DMSO-d6): δ 1.97 (s, 3H), δ 2.19 (s, 3H), δ 4.14-4.18 (m, 2H), δ 5.07-5.11 (m, 1H), δ 6.25-6.30 (m, 1H), δ 6.50-6.54 (m, 1H), δ 6.76-6.80 (m, 1H), δ 7.90 (s, 1H), δ 11.78 (br, 1H), δ 12.07 (br, 1H).

ESIMS m/z: 334 (M+H).

### Example 3

### Production of 5′-O-acetyl-2′,3′-didehydro-2′,3′-dideoxyinosine

To a solution of 5′-O-acetyl-3′-deoxy-3′β-bromoinosine (93 mg, 0.25 mmol) in acetonitrile (2.5 mL) were added triethylamine (0.139 mL, 1.0 mmol) and perfluoro-1-butane-sulfonyl fluoride (0.180 mL, 1.0 mmol) followed by heating at 70° C. for 2 hours with stirring. The resulting solution was cooled down to room temperature and concentrated in vacuo. As a result of purification by chromatography (10 g of silica gel; eluted with dichloromethane-methanol in 32:1), the desired substance (50.6 mg, yield: 73%) was obtained as a white solid.

¹H-NMR (DMSO-d6): δ 1.98 (s, 3H), δ 4.16-4.20 (m, 2H), δ 5.08-5.12 (m, 1H), δ 6.24-6.28 (m, 1H), δ 6.50-6.54 (m, 1H), δ 6.91-6.93 (m, 1H), δ 8.02 (s, 1H), δ 8.09 (s, 1H), δ 8.87 (br, 1H).

ESIMS m/z: 277 (M+H).

### Example 4

### Production of N²,5′-O-diacetyl-2′,3′-didehydro-2′,3′-dideoxy-2-amino-6-chloropurine Riboside

To a solution of N²,5′-O-diacetyl-3′-deoxy-3′β-bromo-2-amino-6-chloropurine riboside (63 mg, 0.14 mmol) in acetonitrile (1.0 mL) were added triethylamine (0.057 mL, 0.4 mmol) and perfluoro-1-butanesulfonyl fluoride (0.072 mL, 0.4 mmol) followed by heating at 70° C. for 2 hours with stirring. The resulting solution was cooled down to room temperature and concentrated in vacuo. As a result of purification by chromatography (20 g of silica gel; eluted with hexane-ethyl acetate in 1:4), the desired substance (47.5 mg, yield: 97%) was obtained as a white solid.

¹H-NMR (CDCl₃): δ 2.08 (s, 3H), δ 2.56 (s, 3H), δ 4.23-4.28 (m, 1H), δ 4.37 (d-d, 1H, J=12.4, 3.6 Hz), δ 5.16-5.23 (m, 1H), δ 6.15 (d, 1H, J=4.8 Hz), δ 6.43 (d-d, J=6.0, 1.6 Hz), δ 7.03-7.09 (m, 1H), δ 8.09 (br, H), δ 8.18 (d, 1H, J=3.6 Hz).

ESIMS m/z: 374 (M+Na).

### Example 5

### Production of 5′-O-acetyl-2′,3′-didehydro-2′,3′-dideoxyadenosine

To a solution of 5′-O-acetyl-3′-deoxy-3′β-bromo-adenosine (186 mg, 0.5 mmol) in acetonitrile (5 mL) were added triethylamine (0.139 mL, 1.0 mmol) and perfluoro-1-butane-sulfonyl fluoride (0.180 mL, 1.0 mmol) followed by heating at 70° C. for 2 hours with stirring. As a result of quantification by means of HPLC, it was confirmed that the desired substance was produced in the resulting solution in an amount of 99 mg (yield: 71%).

### Example 6

### Production of 5′-O-acetyl-2′,3′-didehydro-2′,3′-dideoxyadenosine

To a solution of 5′-O-acetyl-3′-deoxy-3′β-bromo-adenosine (186 mg, 0.5 mmol) in acetonitrile (5 mL) were added triethylamine (0.278 mL, 2.0 mmol) and perfluoro-1-butane-sulfonyl fluoride (0.180 mL, 1.0 mmol) followed by heating at 70° C. for 2 hours with stirring. As a result of quantification by means of HPLC, it was confirmed that the desired substance was produced in the resulting solution in an amount of 125 mg (yield: 91%).

### Example 7

### Production of 5′-O-acetyl-2′,3′-dideoxyadenosine

To a solution of 5′-O-acetyl-2′,3′-didehydro-2′,3′-dideoxyadenosine (27 mg, 0.1 mmol) in methanol (1 mL) was added 5% palladium carbon (containing 50% of water) (2.7 mg) followed by stirring at room temperature for 14 hours in a hydrogen atmosphere. The palladium catalyst was removed from the resulting solution by filtration, and the filtrate was dried in vacuo to give the desired substance (25 mg, yield: 90%) as a white solid.

¹H-NMR (DMSO-d6): δ 1.97 (s, 3H), δ 2.10-2.17 (m, 2H), δ 2.45-2.53 (m, 2H), δ 4.14 (d-d, 1H), J=11.8, 6.0 Hz), δ 4.22 (d-d, 1H, J=11.8, 3.1 Hz), δ 4.28-4.30 (m, 1H), δ 6.25 (d-d, 1H, J=6.8, 3.6 Hz), δ 7.27 (br, 2H), δ 8.15 (s, 1H), δ 8.28 (s, 1H).

ESIMS m/z: 278 (M+H).

### Industrial Applicability

The 2′,3′-didehydro-2′,3′-dideoxypurine nucleoside compounds and the 2′,3′-dideoxypurine nucleoside compounds produced by the method of the present invention are able to be advantageously used as pharmaceuticals such as anti-HIV drugs or intermediate compounds for the synthesis thereof.

Obviously, numerous modifications and variations of the present invention are possible in light of the above teachings. It is therefore to be understood that, within the scope of the appended claims, the invention may be practiced otherwise than as specifically described herein.

All patents and other references mentioned above are incorporated in full herein by this reference, the same as if set forth at length.

1. A method for producing a 2′,3′-didehydro-2′,3′-dideoxypurine nucleoside compound represented by formula (2): 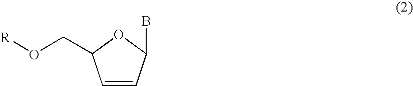
wherein:
R is a protective group for a hydroxyl group; and
B is a purine base,
wherein said method comprises:
(a) treating a 3′-deoxy-3′-halopurine nucleoside compound represented by formula (1): 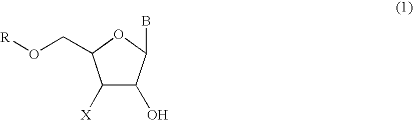
wherein:
X is chlorine atom, bromine atom, or iodine atom; and
R and B are defined above, with at least one perfluoroalkanesulfonyl fluoride in the presence of at least one base, to obtain said 2′,3′-didehydro-2′,3′-dideoxypurine nucleoside compound of formula (2).

2. The method of claim 1, wherein said at least one base comprises at least one tertiary amine.

3. The method of claim 1, wherein said at least one base is selected from the group consisting of trimethylamine, triethylamine, triethylenediamine, N,N-dimethylcyclohexylamine, tetramethylenediamine, N,N,N′,N′-tetramethyl-1,3-butanediamine, N-methylmorpholine, N,N-diethyl-2-methylpiperazine, DBU (1,8-diazabicyclo[5,4,0]undec-7-ene), and mixtures thereof.

4. The method of claim 1, wherein said at least one perfluoroalkanesulfonyl fluoride is selected from the group consisting of perfluorobutanesulfonyl fluoride, perfluorohexanesulfonyl fluoride, perfluorooctanesulfonyl fluoride, and mixtures thereof.

5. A method for producing a 2′,3′-dideoxypurine nucleoside compound represented by formula (3): 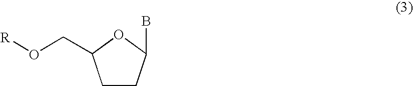
wherein:
R is a protective group for a hydroxyl group; and
B is a purine base,
wherein said method comprises:
(a) treating a 3′-deoxy-3′-halopurine nucleoside compound represented by formula (1): 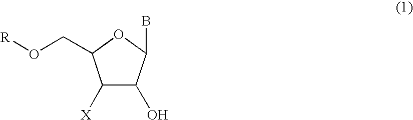
wherein:
X is chlorine atom, bromine atom, or iodine atom; and
R and B are defined above, with at least one perfluoroalkanesulfonyl fluoride in the presence of at least one base, to obtain a 2′,3′-didehydro-2′,3′-dideoxypurine nucleoside compound represented by formula (2): 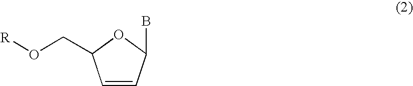
wherein:
R and B are defined above; and
(b) subjecting said 2′,3′-didehydro-2′,3′-dideoxypurine nucleoside compound of formula (2) to catalytic hydrogenation, to obtain said 2′,3′-dideoxypurine nucleoside compound of formula (3).

6. The method of claim 5, wherein said at least one base comprises a tertiary amine.

7. The method of claim 5, wherein said at least one base is selected from the group consisting of trimethylamine, triethylamine, triethylenediamine, N,N-dimethylcyclohexylamine, tetramethylenediamine, N,N,N′,N′-tetramethyl-1,3-butanediamine, N-methylmorpholine, N,N-diethyl-2-methylpiperazine, DBU (1,8-diazabicyclo[5,4,0]undec-7-ene), and mixtures thereof.

8. The method of claim 5, wherein said at least one perfluoroalkanesulfonyl fluoride is selected from the group consisting of perfluorobutanesulfonyl fluoride, perfluorohexanesulfonyl fluoride, perfluorooctanesulfonyl fluoride, and mixtures thereof.

9. A method for producing a 2′,3′-didehydro-2′,3′-dideoxypurine nucleoside compound represented by formula (2′): 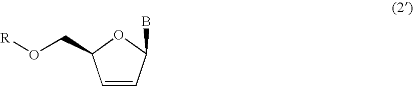
wherein:
R is a protective group for a hydroxyl group; and
B is a purine base,
wherein said method comprises:
(a) treating a 3′-deoxy-3′β′-halopurine nucleoside compound represented by formula (1′): 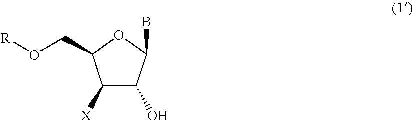
wherein:
X is chlorine atom, bromine atom, or iodine atom; and
R and B are defined above, with at least one perfluoroalkanesulfonyl fluoride in the presence of at least one base, to obtain said 2′,3′-didehydro-2′,3′-dideoxypurine nucleoside compound of formula (2′).

10. The method of claim 9, wherein said at least one base comprises at least one tertiary amine.

11. The method of claim 9, wherein said at least one base is selected from the group consisting of trimethylamine, triethylamine, triethylenediamine, N,N-dimethylcyclohexylamine, tetramethylenediamine, N,N,N′,N′-tetramethyl-1,3-butanediamine, N-methylmorpholine, N,N-diethyl-2-methylpiperazine, DBU (1,8-diazabicyclo[5,4,0]undec-7-ene), and mixtures thereof.

12. The method of claim 9, wherein said at least one perfluoroalkanesulfonyl fluoride is selected from the group consisting of perfluorobutanesulfonyl fluoride, perfluorohexanesulfonyl fluoride, perfluorooctanesulfonyl fluoride, and mixtures thereof.

13. A method for producing a 2′,3′-dideoxypurine nucleoside compound represented by formula (3′): 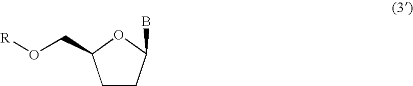
wherein:
R is a protective group for a hydroxyl group; and
B is a purine base,
wherein said method comprises:
(a) treating a 3′-deoxy-3′β′-halopurine nucleoside compound represented by formula (1′): 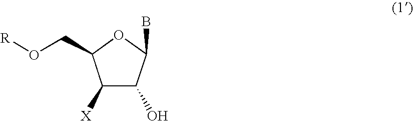
wherein:
X is chlorine atom, bromine atom, or iodine atom; and
R and B are defined above, with at least one perfluoroalkanesulfonyl fluoride in the presence of at least one base, to obtain a 2′,3′-didehydro-2′,3′-dideoxypurine nucleoside compound represented by formula (2′): 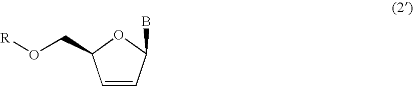
wherein:
R and B are defined above; and
(b) subjecting said 2′,3′-didehydro-2′,3′-dideoxypurine nucleoside compound of formula (2′) to catalytic hydrogenation, to obtain said 2′,3′-dideoxypurine nucleoside compound of formula (3′).

14. The method of claim 13, wherein said at least one base comprises at least one tertiary amine.

15. The method of claim 13, wherein said at least one base is selected from the group consisting of trimethylamine, triethylamine, triethylenediamine, N,N-dimethylcyclohexylamine, tetramethylenediamine, N,N,N′,N′-tetramethyl-1,3-butanediamine, N-methylmorpholine, N,N-diethyl-2-methylpiperazine, DBU (1,8-diazabicyclo[5,4,0]undec-7-ene), and mixtures thereof.

16. The method of claim 13, wherein said at least one perfluoroalkanesulfonyl fluoride is selected from the group consisting of perfluorobutanesulfonyl fluoride, perfluorohexanesulfonyl fluoride, perfluorooctanesulfonyl fluoride, and mixtures thereof.

17. N²,5′-O-Diacetyl-3′-deoxy-3′β-bromo-guanosine represented by the formula (1a): 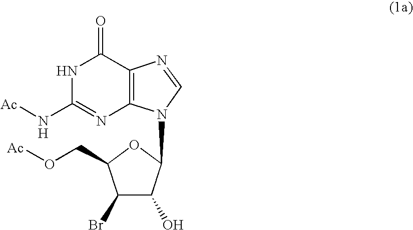
wherein Ac is acetyl group.

18. N²,5′-O-Diacetyl-2′,3′-didehydro-2′,3′-dideoxyguanosine represented by the formula (2a): 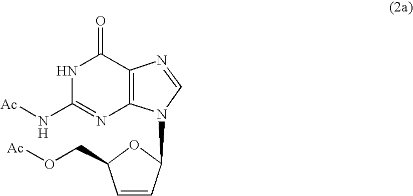
wherein Ac is acetyl group.

19. N²,5′-O-Diacetyl-3′-deoxy-3′β-bromo-2-amino-6-chloropurine riboside represented by the formula (1b): 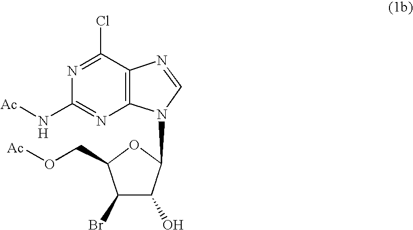
wherein Ac is acetyl group.

20. N²,5′-O-Diacetyl-2′,3′-didehydro-2′,3′-dideoxy-2-amino-6-chloropurine riboside represented by the formula (2b): 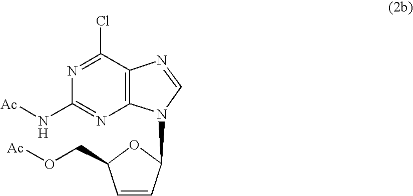
wherein Ac is acetyl group.

21. A method for producing a 2′,3′-didehydro-2′,3′-dideoxypurine nucleoside compound represented by formula (6): 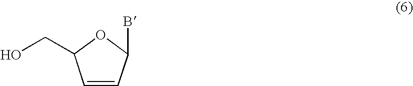
wherein:
B′ is a purine base,
wherein said method comprises:
(a) treating a 3′-deoxy-3′-halopurine nucleoside compound represented by formula (1): 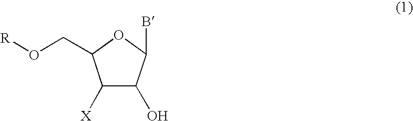
wherein:
X is chlorine atom, bromine atom, or iodine atom;
R is a protecting group; and
B′ is defined above,
with at least one perfluoroalkanesulfonyl fluoride in the presence of at least one base, to obtain a 2′,3′-didehydro-2′,3′-dideoxypurine nucleoside compound represented by formula (2): 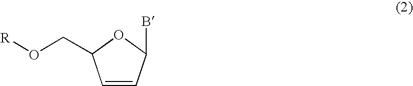
wherein
R and B′ are defined above; and
(b) subjecting said 2′,3′-didehydro-2′,3′-dideoxypurine nucleoside compound of formula (2) to de-protection of R, and if necessary to at least one of protection, de-protection, and modification of a group at 2-position and/or 6-position of the purine base.

22. A method for producing a 2′,3′-didehydro-2′,3′-dideoxypurine nucleoside compound represented by formula (6′): 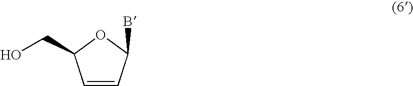
wherein:
B′ is a purine base,
wherein said method comprises:
(a) treating a 3′-deoxy-3′-halopurine nucleoside compound represented by formula (1′): 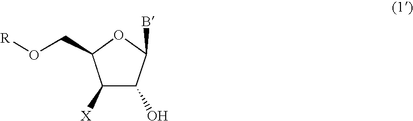
wherein
X is chlorine atom, bromine atom, or iodine atom;
R is a protecting group; and
B′ is defined above,
with at least one perfluoroalkanesulfonyl fluoride in the presence of at least one base, to obtain a 2′,3′-didehydro-2′,3′-dideoxypurine nucleoside compound represented by formula (2′) 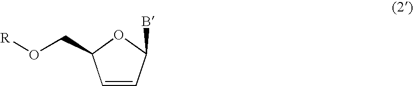
wherein:
R and B′ are defined above; and
(b) subjecting said 2′,3′-didehydro-2′,3′-dideoxypurine nucleoside compound of formula (2′) to de-protection of R, and if necessary to at least one of protection, de-protection, and modification of a group at 2-position and/or 6-position of the purine base.

23. A method for producing a 2′,3′-dideoxypurine nucleoside compound represented by formula (7): 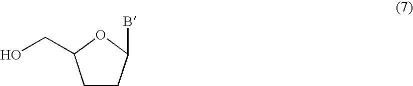
wherein:
B′ is a purine base,
wherein said method comprises:
(a) treating a 3′-deoxy-3′-halopurine nucleoside compound represented by formula (1): 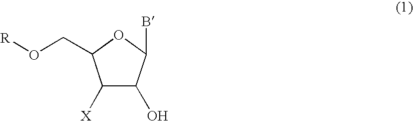
wherein:
X is chlorine atom, bromine atom, or iodine atom;
R is a protecting group; and
B′ is defined above,
with at least one perfluoroalkanesulfonyl fluoride in the presence of at least one base, to obtain a 2′,3′-didehydro-2′,3′-dideoxypurine nucleoside compound represented by formula (2): 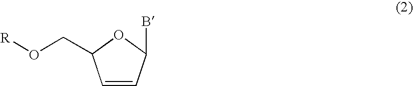
wherein
R and B′ are defined above;
(b) subjecting said 2′,3′-didehydro-2′,3′-dideoxypurine nucleoside compound of formula (2) to catalytic hydrogenation, to obtain a 2′,3′-dideoxypurine nucleoside compound of formula (3): 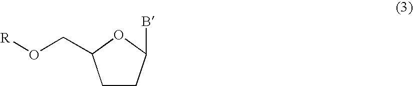
wherein:
R and B′ are defined above; and
(c) subjecting said 2′,3′-dideoxypurine nucleoside compound of formula (3) to de-protection of R, and if necessary to at least one of protection, de-protection, and modification of a group at 2-position and/or 6-position of the purine base.

24. A method for producing a 2′,3′-dideoxypurine nucleoside compound represented by formula (7′) 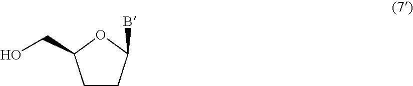
wherein:
B′ is a purine base,
wherein said method comprises:
(a) treating a 3′-deoxy-3′-halopurine nucleoside compound represented by formula (1′): 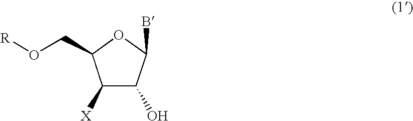
wherein:
X is chlorine atom, bromine atom, or iodine atom;
R is a protecting group; and
B′ is defined above,
with at least one perfluoroalkanesulfonyl fluoride in the presence of at least one base, to obtain a 2′,3′-didehydro-2′,3′-dideoxypurine nucleoside compound represented by formula (2′): 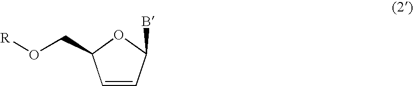
wherein:
R and B′ are defined above;
(b) subjecting said 2′,3′-didehydro-2′,3′-dideoxypurine nucleoside compound of formula (2′) to catalytic hydrogenation, to obtain a 2′,3′-dideoxypurine nucleoside compound of formula (3′): 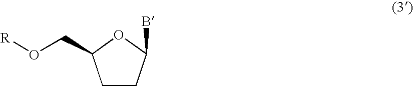
wherein:
R and B′ are defined above; and
(c) subjecting said 2′,3′-dideoxypurine nucleoside compound of formula (3′) to de-protection of R, and if necessary to at least one of protection, de-protection, and modification of a group at 2-position and/or 6-position of the purine base.
